(19) United States
(12) Patent Application Publication
(10) Pub. No.: US 2022/0241524 A1
(43) Pub. Date: Aug. 4, 2022

(54) AEROSOL FORMULATION, CANISTER, AND INHALER CONTAINING THE FORMULATION, AND METHOD OF USE

(71) Applicant: KINDEVA DRUG DELIVERY L.P., Woodbury, MN (US)

(72) Inventors: Alexander D. Slowey, Nottingham (GB); Sarah E. Wrigglesworth, Leicestershire (GB)

(73) Assignee: KINDEVA DRUG DELIVERY L.P., Woodbury, MN (US)

(21) Appl. No.: 17/620,325

(22) PCT Filed: Mar. 3, 2020
(86) PCT No.: PCT/IB2020/051791

§ 371 Date: Dec. 17, 2021

Related U.S. Application Data
(60) Provisional application No. 62/873,397, filed on Jul. 12, 2019.

(51) Int. Cl. A61M 15/00; A61K 31/439; A61K 31/137; A61K 47/32; A61K 9/00

(52) U.S. Cl. CPC A61M 15/0066; A61K 31/439; A61M 2205/0238; A61K 47/32; A61K 9/008; A61K 31/137

(57) ABSTRACT

A pressurized canister coated with poly(fluoroalkylene) polymer or a copolymer of poly(fluoralkylenes) and containing a formulation comprising one or more active pharmaceutical ingredients, PVP, and a propellant, as well as methods of using the same.

### BACKGROUND

Aerosol formulations, particularly those containing one or more active pharmaceutical ingredients, are known in the art to also contain propellants and excipients.

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art, and should be afforded the meaning that a person of ordinary skill in the art would have ascribed to them.

The terms “common,” “typical,” and “usual,” as well as “commonly,” “typically,” and “usually” are used herein to refer to features that are often employed in the invention and, unless specifically used with reference to the prior art, are not intended to mean that the features are present in the prior art, much less that those features are common, usual, or typical in the prior art.

The term “active pharmaceutical ingredient” is intended to include both free compounds as well as pharmaceutically acceptable salts, hydrates, and solvates thereof. Salts of hydrates or solvates are also included, so long as they are pharmaceutically acceptable. When the name of a particular active pharmaceutical ingredient is used, it is likewise intended to include pharmaceutically acceptable salts, hydrates, and solvates thereof, as well as salts of hydrates or solvates so long as they are pharmaceutically acceptable. If the free compound or a particular salt, hydrate, solvate, or the like is specifically called for, it is identified as such in this disclosure.

The term “poly(ethylene glycol)” is used to refer to a polymer having the repeat unit —O—(CH₂)₂—. Depending on the way in which it is produced, poly(ethylene oxide) may have the same chemical structure, in which case it is encompassed by the term poly(ethylene glycol).

The “number average degree of polymerization” is the average number of structural units (polymerized monomer units or repeat units) per polymer chain.

In this disclosure, the percent (%) concentration of components in a formulation is provided as a weight percent unless otherwise specified.

Fluoroalkanes are alkanes wherein at least some of the hydrogen atoms are replaced by fluoride.

Perfluoroalkanes are fluoroalkanes wherein essentially all of the hydrogen atoms are replaced by fluorine, but still allow the possibility that a small number of hydrogen atoms may be replaced by bromine or iodine instead of fluorine.

Fluoroalkanes and perfluoroalkanes may be referred to with carbon numbers to indicate the number of carbon atoms; in the event that a polymer or copolymer is referred to, the carbon numbers refer to the number of carbon atoms in the monomer or monomers from which the polymer was made.

“Trace amount” or “trace amounts” refers to an amount of a component that is unavoidably or unintentionally present, and the like, for example as amounts of contaminants, byproducts or a chemical reaction or industrial process (for example, filling and pressurizing a canister), minor components of industrially available materials, amounts that are not conveniently removable by common purification methods, and so forth. Trace amounts of materials in a formulation are limited to those amounts that do not have any appreciable effect on the properties of the formulation.

“Alcohol” refers to alcohol solvents or dispersants, typically ethanol but also including methanol, propanol, butanol, and the like. When the chemical moiety —OH is referred to, the term “alcohol moiety” is used.

“PVP” is used as an abbreviation for poly(vinyl pyrrolidone), which is sometimes known in the art by its various trade designations, such as povidone The PVP used in this disclosure is typically not crosslinked or only lightly crosslinked such that the PVP is soluble, or in some cases highly dispersable, in the formulations described herein; crospovidone is rarely used in the context of this disclosure. In particular cases, the PVP is soluble in the formulation.

Delivery of formulations containing active pharmaceutical ingredients by inhalation can be performed using inhalers with valves, such as metering valves. Metering valves are valves that regulate the amount of formulation that passes out of the inhaler and is delivered to the patient.

In order to deliver the desired dose of active pharmaceutical ingredient to the patient, it is not only necessary that the same amount of formulation pass through the valve each time the valve is opened to deliver a dose, but it is also necessary that the concentration of active pharmaceutical ingredient in the formulation that passes out of the container, through the valve, and to the patient be the same as the concentration of active pharmaceutical ingredient in the container.

A problem recognized in this disclosure is that active pharmaceutical ingredients can be deposited on the interior components of an inhaler, such as a metered dose inhaler. Particularly, active pharmaceutical ingredients can be deposited on the interior of the canister, on the valve, or both. Excessive deposition is not acceptable in an inhaler and is particularly unacceptable in a metered dose inhaler.

It should be noted that an acceptable solution to this problem need not eliminate all deposition of the one or more pharmaceutically active agents from the inhaler. Instead, an acceptable solution would maintain the amount of the one or more pharmaceutically active agents that is deposited on the valve and canister to reasonably low levels.

Another problem is that there are currently no acceptable metered dose inhalers of certain inhalable active pharmaceutical ingredients, in particular umeclidinium and vilanterol, and more particularly umeclidinium bromide and vilanterol trifenatate. A dry powder inhaler of umeclidinium bromide and vilanterol trifenatate is available from GlaxoSmithKline under the trade designation Anoro Ellipta. However, dry powder inhalers are not acceptable for all patients because they rely solely on the power of the patient's inhalation to deliver the drug, and many patients who require inhaled medicines are not able to inhale deeply enough or with enough power to receive drugs from a dry powder inhaler. Thus, another problem to be solved relates to a metered dose inhaler, and pressurized canister therefor, that can be used to deliver umeclidinium or vilanterol or both, and particularly umeclidinium bromide and vilanterol trifenatate.

Briefly, a solution lies in the combination of a formulation having one or more active pharmaceutical ingredients, one or more propellants, and PVP within a pressurized canister, wherein the interior of the pressurized canister is coated with a poly(fluoroalkylene) polymer or a copolymer of poly(fluoralkylenes). It has been surprisingly shown that when the foregoing is used the actual dose is much closer to the theoretical dose than with other excipient and pressurized canister coating combinations; it is also an improvement over excipient-free formulations.

The one or more active pharmaceutical ingredients can in principle be any active pharmaceutical ingredients, but most typically selected from active pharmaceutical ingredients that are suitable for delivery by inhalation. In some cases, the one or more active pharmaceutical ingredients can include one or more of albuterol, levabuterol, formoterol, glycopyrrate, ciclesonide, mometasone, fluticasone, formaterol, ipratropium, beclomethasone, epinephrine, tiotropium, nicotine, umeclidimium, and vilanterol. In some cases, the one or more active pharmaceutical ingredients can include one or more active pharmaceutical ingredients that have at least one amine moiety, at least one alcohol moiety, or at least one amine moiety and at least one alcohol moiety. In some cases, the one or more active pharmaceutical ingredients can include one or more active pharmaceutical ingredients that have at least one amine moiety and at least one alcohol moiety. In specific cases, the one or more active pharmaceutical ingredients comprise umeclidinium or vilanterol. In more specific cases, the one or more active pharmaceutical ingredients comprise umeclidinium and vilanterol. The one or more active pharmaceutical ingredients can be present in the formulation either as a suspension, typically of micronized particles of the one or more active pharmaceutical ingredients, or they can be dissolved in the formulation. In most cases, the one or more active pharmaceutical ingredients are present as suspensions in the formulation. The concentration of any of the one or more active pharmaceutical ingredients in the formulation can be any concentration that provides a suitable dosage of the particular active pharmaceutical ingredient to the patient.

The propellant can be any propellant suitable for use in an inhaler, such as a metered dose inhaler, but is most commonly a hydrofluorocarbon propellant. Examples include HFC-227, HFC-152a, and HFC-134a, as well as combinations of two or more of the foregoing. HFC-227 is most commonly used.

Different types of PVP may be characterized by their viscosity in solution, expressed as a K-value (see European Pharmacopoeia, 5th ed., 2004, vol. 2, page 2289). The K-value of the PVP used can be 10 or greater, 15 or greater, or even 20 or greater The K value of the PVP used can be 150 or less, 100 or less, 80 or less, 40 or less, or even 35 or less. In one embodiment the K-value of the PVP is between 20 and 35. Suitable polyvinylpyrrolidones are PVP(K25), PVP(K30), Povidone K30, PVP(K29/32), PVP(K90), PVP(K120), PVP (C15), PVP(C30) or PVP/17PF.

The weight average molecular weight of the PVP used (in Daltons) can be 2,000 or greater, 4,000 or greater, 5,000 or greater, 7,500 or greater, or even 10,000 or greater. The weight average molecular weight of the PVP used (in Daltons) can be 1,500,000 or less, 1,000,000 or less, 500,000 or less, 100,0000 or less, 60,000 or less, 50,000 or less, or even 40,000 or less.

The concentration of the PVP in the formulation can be any amount sufficient to decrease the amount of active pharmaceutical ingredient that is deposited on the canister of valve of the inhaler in comparison to the amount that would be deposited in the absence of PVP. Most commonly, the PVP will be present in a concentration of about 0.001% to about 1% of the formulation. Exemplary concentrations can be about 0.001% or greater, 0.005% or greater, 0.0075% or greater, 0.01% or greater, 0.02% or greater, 0.03% or greater, 0.04% or greater, 0.05% or greater, 0.06% or greater, 0.07% or greater, 0.08% or greater, or 0.09% or greater. Exemplary concentrations can be 1% or less, 0.9% or less, 0.8% or less, 0.7% or less, 0.6% or less, 0.5% or less, 0.45% or less, 0.4% or less, 0.35% or less, 0.3% or less, 0.29% or less, 0.28% or less, 0.27% or less, 0.26% or less, 0.25% or less, 0.24% or less, 0.23% or less, 0.22% or less, 0.21% or less, 0.2% or less, 0.19% or less, 0.18% or less, 0.17% or less, 0.16% or less, 0.15% or less, 0.14% or less, 0.13% or less, 0.12% or less, or 0.11% or less. A concentration can be from about 0.005% to about 0.05%. In a particular case the concentration can be 0.01%.

Particular formulation can be free of more than trace amounts of components other than the one or more active pharmaceutical ingredients, the one or more propellants, and the PVP. Some particular formulations are free of more than trace amounts of alcohols, particularly ethanol. Some particular formulations are free of more than trace amounts of water. Some particular formulations are free of more than trace amounts of water and alcohol, such as ethanol. Some particular formulations are free of more than trace amounts of surfactant (other than the PVP).

There are two coatings that can be used. The first coating that can be used is a poly(fluoroalkylene) polymer or a copolymer of poly(fluoralkylenes), most commonly a copolymer of poly(fluoroalkylenes). The fluoroalkylenes used in the polymer or copolymer are typically C2-C10 fluoroalkanes. The fluoroalkylenes used I the polymer or copolymer are most commonly Most commonly, a copolymer of poly(fluoroalkylenes) is used. A typical copolymer is a copolymer of a C2-C4 fluoroalkylene and a C3-C6 fluoroalkylene. Most commonly, the copolymer is a copolymer of hexafluoropropylene and tetrafluoroethylene, which is sometimes referred to as FEP.

The second coating that can be used is the condensation product of two layers. The first layer is a primer layer comprising a silane having one or more reactive silane groups. The second layer, which is disposed on the first layer, comprises an at least partially fluorinated compound having at least one reactive silane group.

The first layer (primer layer) is disposed directly on the inhaler components, such as the interior of the canister, the valve, or both. The second layer is a coating layer comprising an at least partially fluorinated compound having at least one reactive silane groups, most typically one or more of hydrolysable silane group or hydroxysilane group. The second layer is deposited on the first layer, such that the reactive silane groups of the first layer can undergo a chemical reaction, such as a condensation reaction, with the reactive silane groups of the second layer.

The at least one reactive silane group may be of formula Si(R⁰)ₙX₃₋ₙ, wherein R⁰ is a substantially non-hydrolysable group, X is a hydrolysable or hydroxy group and n is 0, 1 or 2.

The first layer (primer layer) is typically a silane having two or more reactive silane groups separated by an organic linker. In particular cases the silane having two or more reactive silane groups is of formula

X₃₋ₘ(R¹)ₘSi-Q-Si(R²)ₖX₃₋ₖ

wherein R¹ and R² are independently selected univalent groups such as C1-C4 alkyl, X is a hydrolysable or hydroxy group, m and k are independently 0, 1, or 2 and Q is a divalent organic linking group.

Usually, Q will comprise a 1 to 12 atom chain, more usually a substituted or unsubstituted C₂ to C₁₂ hydrocarbyl chain. Preferably, Q comprises a substituted or unsubstituted C₂ to C₁₂ alkyl chain.

Useful examples of silanes having two or more reactive silane groups include one or a mixture of two or more of 1,2-bis(trialkoxysilyl) ethane, 1,6-bis(trialkoxysilyl) hexane, 1,8-bis(trialkoxysilyl) octane, 1,4-bis(trialkoxysilylethyl)benzene, bis(trialkoxysilyl)itaconate, and 4,4′-bis(trialkoxysilyl)-1,1′-diphenyl, wherein any trialkoxy group may be independently trimethoxy or triethoxy.

Q may comprise a chain substituted by one or more atoms of N, O, and/or S. Thus, further examples of the silane having two or more reactive silane groups include one or a mixture of two or more of bis(trialkoxysilylpropyl)amine; bis (3-trialkoxysilylpropyl) ethylenediamine; bis (3-trialkoxysilylpropyl) n-methylamine; bis[3-(trialkoxysilyl)propyl]fumarate and N, N-bis (3-trialkoxysilylmethyl) allylamine, wherein any trialkoxy group may be independently trimethoxy or triethoxy.

In particular embodiments, the silane is such that Q may be of formula —(CH₂)ᵢ-A-(CH₂)ⱼ— wherein A is NRⁿ, O, or S; i and j are independently 0, 1, 2, 3 or 4 and wherein Rⁿ is H or C₁ to C₄ alkyl. Even more preferably, Q may be of formula —(CH₂)ᵢ—NH—(CH₂)ⱼ— and i and j are each independently 1, 2, 3 or 4. Most preferably i and j are each 3.

A particular second layer can be a perfluoropolyether silane according to Formula Ia in which R^{f} comprises from 20 to 40 linked repeating units confer additional lubricity compared to those with fewer repeating units, and when these are assembled with other components to make up valves, the valves have lower actuation forces.

Formula Ia is:

R^{f}[Q¹-[C(R)₂—Si(Y)₃₋ₓ(R^{1a})ₓ]_{y}]_{z} Ia

wherein:

R¹ is a monovalent or multivalent polyfluoropolyether moiety;

Q¹ is an organic divalent or trivalent linking group;

each R is independently hydrogen or a C1-4 alkyl group;

each Y is independently a hydrolysable group;

R^{1a} is a C1-8 alkyl or phenyl group;

x is 0 or 1 or 2;

y is 1 or 2; and

z is 1, 2, 3, or 4.

In some particular cases of Formula Ia, x, y, and z are each 1.

In some particular cases of Formula Ia, Y is O—

A more particular first layer is a perfluoropolyether silane of Formula Ib.

R^{f}[Q¹-[C(R)₂—Si(O—)₃₋ₓ(R^{1a})ₓ]_{y}]_{z} Ib

wherein:

R^{f} is a monovalent or multivalent polyfluoropolyether segment;

Q¹ is an organic divalent or trivalent linking group;

each R is independently hydrogen or a C1-4 alkyl group; and

R^{1a} is a C1-8 alkyl or phenyl group.

The second layer may comprise a polyfluoroether silane, in particular a polyfluoropolyether silane. More particularly, the polyfluoroether silane may be a perfluorinated polyfluoroether moiety, even more particularly the polyfluoroether silane may be a perfluorinated polyfluoropolyether silane.

The polyfluoropolyether silane may be of formula

R^{f}Q¹ᵥ[Q²_{w}-[C(R⁴)₂—Si(X)₃₋ₓ(R⁵)ₓ]_{y}]_{z}

wherein:

R^{f} is a polyfluoropolyether moiety;

Q¹ is a trivalent linking group;

each Q² is an independently selected organic divalent or trivalent linking group;

each R⁴ is independently hydrogen or a C₁₋₄ alkyl group;

each X is independently a hydrolysable or hydroxyl group;

R⁵ is a C₁₋₈ alkyl or phenyl group;

v and w are independently 0 or 1, x is 0 or 1 or 2; y is 1 or 2; and z is 2, 3, or 4.

The polyfluoropolyether moiety R^{f} may comprise perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted and wherein for repeating units including Z the number of carbon atoms in sequence is at most 6.

In particular, n may be an integer from 1 to 4, more particularly from 1 to 3. For repeating units including Z the number of carbon atoms in sequence may be at most four, more particularly at most 3. Usually, n is 1 or 2 and Z is an —CF₃ group, more wherein z is 2, and R^{f} is selected from the group consisting of —CF₂O(CF₂O)ₘ(C₂F₄O)ₚCF₂—, —CF(CF₃)O(CF(CF)CF₂O)ₚCF(CF₃)—, —CF₂O(C₂F₄O)ₚCF₂—, —(CF₂)₃O(C₄F₈O)ₚ(CF₂)₃—, —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

The first layer or second layer can be applied, for example to the interior of the canister or the valve, by any suitable method. Independently of the application step used for the second layer, the first layer can be applied by spraying, dipping, rolling, brushing, spreading or flow coating, in particular by spraying or dipping. Independently of the application step used for the first layer, the second layer can be applied by spraying, dipping, rolling, brushing, spreading or flow coating, in particular by spraying or dipping.

In any of the above-mentioned ways of applying the first layer or the second layer, a coating liquid is usually used. Typically, the coating liquid comprises an alcohol or a hydrofluoroether.

If the coating liquid is an alcohol, preferred alcohols are C₁ to C₄ alcohols, in particular, an alcohol selected from ethanol, n-propanol, or iso-propanol or a mixture of two or more of these alcohols.

If the coating liquid is an hydrofluoroether, it is preferred if the coating solvent comprises a C₄ to C₁₀ hydrofluoroether. Generally, the hydrofluoroether will be of formula

C_{g}F_{2g+1}OCₕH₂ₕ₊₁

wherein g is 2, 3, 4, 5, or 6 and his 1, 2, 3 or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

A cross-linking agent can be applied along with the first layer, the second layer, or both the first and second layers. The cross-linking agent may comprise a compound selected from group consisting of tetramethoxysilane; tetraethoxysilane; tetrapropoxysilane; tetrabutoxysilane; methyl triethoxysilane; dimethyldiethoxysilane; octadecyltriethoxysilane; 3-glycidoxy-propyltrimethoxysilane; 3-glycidoxy-propyltriethoxysilane; 3-aminopropyl-trimethoxysilane; 3-aminopropyl-triethoxysilane; bis (3-trimethoxysilylpropyl) amine; 3-aminopropyl tri(methoxyethoxyethoxy) silane; N (2-aminoethyl)3-aminopropyltrimethoxysilane; bis (3-trimethoxysilylpropyl) ethylenediamine; 3-mercaptopropyltrimethoxysilane; 3-mercaptopropyltriethoxysilane; 3-trimethoxysilyl-propylmethacrylate; 3-triethoxysilypropylmethacrylate; bis (trimethoxysilyl) itaconate; allyltriethoxysilane; allyltrimethoxysilane; 3-(N-allylamino)propyltrimethoxysilane; vinyltrimethoxysilane; vinyltriethoxysilane; and mixtures thereof.

Before applying the first layer, a pre-treatment step may be employed. The pre-treatment step will typically comprise cleaning the surface with a cleaning liquid. A particularly useful cleaning liquid comprises a hydrofluoroether, such as HFE72DE, an azeotropic mixture of about 70% w/w trans-dichloroethylene; 30% w/w of a mixture of methyl and ethyl nonafluorobutyl and nonafluoroisobutyl ethers.

After application of the first layer, it may be desirable to cure the first layer. Curing can be effected by evaporating the coating liquid in the presence of humidity, because water can promote curing of the first layer. Heat can also aid promote curing of the first layer. When heat is applied, the temperature should be low enough as to not to deform the valve or canister, which are often (though not always) made of plastics.

Notably, curing of the first layer is not always required because a coating with the desired properties can be formed by applying the second layer directly over an uncured first layer.

Either of the two coatings described above can be used on the interior of the canister, on the valve, or both. When either of the two coatings described above are applied to the valve, they are most often applied to the valve stem. It is also possible to use one coating on the canister and the other on the valve. Specifically, it is possible to coat the interior the canister and the valve with any of the poly(fluoroalkylene) polymer or a copolymer of poly(fluoralkylenes) described above. It is also possible to coat the interior of the canister with any of the poly(fluoroalkylene) polymer or a copolymer of poly(fluoralkylenes) described above and the valve with any of the two-component coatings described above. It is also possible to coat the valve with any of the poly(fluoroalkylene) polymer or a copolymer of poly(fluoralkylenes) described above and the interior of the canister with first layer comprising a silane having one or more reactive silane groups and a second layer deposited on the first layer and comprising an at least partially fluorinated compound having at least one reactive silane groups. It is also possible to coat the interior of the canister and the valve with that coating.

An inhaler can contain a pressurized canister as described herein. Typically, inhalers will also include a valve. The valve is typically in communication with an actuator such that when the actuator is actuated at least a part of the formulation is released from the inhaler. Most often the valve is a metering valve. Metering valves, sometimes referred to as metered dose valves, are known, and any suitable metering valve can be used. Suitable metering valves include those that are able to release a volume of formulation with a pharmaceutically effective amount of active pharmaceutical agent, and that do not chemically interact with the components of the formulation. At least a portion of the metering valve can be coated with a poly(fluoroalkylene) polymer or a copolymer of poly(fluoralkylenes), such as any of the poly(fluoroalkylene) polymer or a copolymer of poly(fluoralkylenes) described above with respect to the coating of the canister. This is not required, and other coatings or in some cases no coatings can also be used.

A method of actuating an inhaler, such as any inhaler described herein, is disclosed. The method can comprise actuating an actuator for a sufficient period of time to release at least a portion of the formulation from the pressurized canister. When the method is performed with umeclidinium bromide, a single actuation of the inhaler can release 55.2-74.8 micrograms of umeclidinium bromide from the inhaler. In some embodiments of the method, a single actuation of the inhaler releases 58.5-71.5 micrograms of umeclidinium bromide from the inhaler. When the method is employed with vilanterol trifenatate, a single actuation of the inhaler can release 29.8-40.3 micrograms of vilanterol trifenatate from the inhaler. In some embodiments of the method, a single actuation of the inhaler releases 31.5-39.0 micrograms of vilanterol trifenatate from the inhaler. It should be noted that the term “a single actuation” should not be understood to require not every actuation releases the above-mentioned amounts of pharmaceutically active agent or agents. For example, one or more initial or priming actuation or actuations may release less than the specified amounts.

A method of administering a formulation, such as the formulations disclosed herein, is disclosed. The method can comprise actuating the actuator for a sufficient time to release at least a portion of a formulation as described herein from the pressurized canister and inhaling at least a portion of the formulation. The method can include administering a pharmaceutically acceptable amount of umeclidinium, vilanterol, or a combination thereof. When the method is performed with umeclidinium bromide, a single actuation of the inhaler can release 55.2-74.8 micrograms of umeclidinium bromide from the inhaler. In some embodiments of the method, a single actuation of the inhaler releases 58.5-71.5 micrograms of umeclidinium bromide from the inhaler. When the method is employed with vilanterol trifenatate, a single actuation of the inhaler can release 29.8-40.3 micrograms of vilanterol trifenatate from the inhaler. In some embodiments of the method, a single actuation of the inhaler by the method releases 31.5-39.0 micrograms of vilanterol trifenatate from the inhaler. It should be noted that the term “a single actuation” should not be understood to require not every actuation releases the above-mentioned amounts of pharmaceutically active agent or agents. For example, one or more initial or priming actuation or actuations may release less than the specified amounts.

A method of actuating an inhaler, such as any inhaler described herein, is disclosed. The method can comprise actuating an actuator for a sufficient period of time to release at least a portion of the formulation from the pressurized canister. When the method is performed with umeclidinium bromide, a single actuation of the inhaler can release a metered dose of 63.0-85.3 micrograms of umeclidinium bromide from the valve. In some embodiments of the method, a single actuation of the inhaler releases 66.8-81.6 micrograms of umeclidinium bromide from the valve. When the method is employed with vilanterol trifenatate, a single actuation of the inhaler can release a metered dose of 34.0-46.0 micrograms of vilanterol trifenatate from the valve. In some embodiments of the method, a single actuation of the inhaler releases 36.0-44.0 micrograms of vilanterol trifenatate from the valve. It should be noted that the term “a single actuation” should not be understood to require not every actuation releases the above-mentioned amounts of pharmaceutically active agent or agents. For example, one or more initial or priming actuation or actuations may release less than the specified amounts.

A method of administering a formulation, such as the formulations disclosed herein, is disclosed. The method can comprise actuating the actuator for a sufficient time to release at least a portion of a formulation as described herein from the pressurized canister and inhaling at least a portion of the formulation. The method can include administering a pharmaceutically acceptable amount of umeclidinium, vilanterol, or a combination thereof. When the method is performed with umeclidinium bromide, a single actuation of the inhaler can release a metered dose of 63.0-85.3 micrograms of umeclidinium bromide from the valve. In some embodiments of the method, a single actuation of the inhaler releases 66.8-81.6 micrograms of umeclidinium bromide from the valve. When the method is employed with vilanterol trifenatate, a single actuation of the inhaler can release a metered dose of 34.0-46.0 micrograms of vilanterol trifenatate from the valve. In some embodiments of the method, a single actuation of the inhaler releases 36.0-44.0 micrograms of vilanterol trifenatate from the valve. It should be noted that the term “a single actuation” should not be understood to require not every actuation releases the above-mentioned amounts of pharmaceutically active agent or agents. For example, one or more initial or priming actuation or actuations may release less than the specified amounts.

For any of the methods of actuating an inhaler and methods of administering a formulation that are described above, a single actuation of the inhaler can release 0.11-0.15 micromoles of umeclidinium or a pharmaceutically acceptable salt thereof from the inhaler. In some embodiments of the methods, a single actuation of the inhaler releases 0.12-0.14 micromoles of umeclidinium or a pharmaceutically acceptable salt thereof from the inhaler.

For any of the methods of actuating an inhaler and methods of administering a formulation that are described above, a single actuation of the inhaler can release 0.038-0.052 micromoles of vilanterol or a pharmaceutically acceptable salt thereof from the inhaler. In some embodiments of the methods, a single actuation of the inhaler releases 0.040-0.050 micromoles of vilanterol or a pharmaceutically acceptable salt thereof from the inhaler.

For any of the methods of actuating an inhaler and methods of administering a formulation that are described above, a single actuation of the inhaler can release a metered dose of 0.12-0.17 micromoles of umeclidinium or a pharmaceutically acceptable salt thereof from the valve. In some embodiments of the methods, a single actuation of the inhaler releases 0.13-0.16 micromoles of umeclidinium or a pharmaceutically acceptable salt thereof from the valve.

For any of the methods of actuating an inhaler and methods of administering a formulation that are described above, a single actuation of the inhaler can release a metered dose of 0.044-0.060 micromoles of vilanterol or a pharmaceutically acceptable salt thereof from the valve. In some embodiments of the methods, a single actuation of the inhaler releases 0.046-0.056 micromoles of vilanterol or a pharmaceutically acceptable salt thereof from the valve.

For any of the methods of actuating an inhaler and methods of administering a formulation that are described above, a single actuation of the inhaler can release 46.7-63.3 micrograms of umeclidinium from the inhaler. In some embodiments of the methods, a single actuation of the inhaler releases 49.5-60.5 micrograms of umeclidinium from the inhaler.

For any of the methods of actuating an inhaler and methods of administering a formulation that are described above, a single actuation of the inhaler can release 18.7-25.3 micrograms of vilanterol from the inhaler. In some embodiments of the methods, a single actuation of the inhaler releases 19.8-24.2 micrograms of vilanterol from the inhaler.

For any of the methods of actuating an inhaler and methods of administering a formulation that are described above, a single actuation of the inhaler can release a metered dose of 53.0-71.9 micrograms of umeclidinium from the valve. In some embodiments of the methods, a single actuation of the inhaler releases 56.2-68.8 micrograms of umeclidinium from the valve.

For any of the methods of actuating an inhaler and methods of administering a formulation that are described above, a single actuation of the inhaler can release a metered dose of 21.2-28.8 micrograms of vilanterol from the valve. In some embodiments of the methods, a single actuation of the inhaler releases 22.5-27.5 micrograms of from the valve.

### EXAMPLES

1,1,1,2-tetrafluoroethane (HFC-227) was obtained from Mexichem UK (Runcorn, UK). Vilanterol trifenatate and umeclidinium bromide were obtained from Hovione (Loures, Portugal). Polyvinylpyrrolidone (PVP KOLLIDON 25) was obtained from the Sigma-Aldrich Company (St. Louis, Mo.).

### Example 1

Each metered dose inhaler (MDI) was prepared using a 16 mL aluminum canister coated with FEP (IntraPac International, Mooresville, N.C., USA); a 63 microliter 3M retention type valve with a PBT (polybutylene terephthalate) stem and EPDM (ethylene-propylene diene terpolymer elastomer) diaphragm seal (3M Corporation); and an actuator with a 0.25 mm exit orifice diameter and 0.8 mm jet length. The bottle emptier, tank, spring, and ferrule components of the valves were coated with a fluoropolymer coating according to the general process described in Example 2 of U.S. Patent Application Publication 2017/0152396 A1 (hereby incorporated by reference). The formulation used was umeclidinium bromide (1.237 mg/mL), vilanterol trifenatate (0.667 mg/mL), and PVP (0.01 weight percent) in HFC-227 propellant. In the process, the HFC-227 propellant was dispensed into a batching vessel maintained at negative 60° C. and then PVP was added to the vessel and dispersed to form a mixture. Umeclidinium bromide and vilanterol trifenatate were then added to the vessel and the mixture was high shear mixed to create a homogenous suspension (Silverson mixer, Silverson Machine Ltd., Chesham, UK). The canisters were cold filled with the suspension using a timer-controlled solenoid valve. The inhalers were stored for 6 weeks under ambient conditions prior to testing.

Each finished inhaler was prepared to deliver targeted amounts of 74.2 micrograms umeclidinium bromide ex-valve per actuation and 40 micrograms of vilanterol trifenatate ex-valve per actuation.

The targeted delivery amounts were also calculated to represent the corresponding amount (micrograms) of umeclidinium delivered and the corresponding amount of vilanterol delivered. The calculated amounts were determined by the following equations (MW is an abbreviation for molecular weight):

$$umeclidinium ( µg / actuation ) = [ umeclidinium bromide ( µg / actuation ) ] \times [ MW umeclidinium \div MW umeclidinium bromide ] \\ vilanterol ( µg / actuation ) = [ vilanterol trifenatate ( µg / actuation ) ] \times [ MW vilanterol \div MW vilanterol trifenatate ]$$

Based on the equations, each finished inhaler was calculated to deliver targeted amounts of 62.5 micrograms umeclidinium ex-valve per actuation and 25 micrograms of vilanterol ex-valve per actuation.

### Comparative Example A

MDIs were prepared using the same method as described in Example 1 with the exception that the PVP was not included in the formulation.

### Example 2. Assay for Measuring Deposition of Umeclidinium and Vilanterol on the Valve Assembly

MDIs were prepared and stored as in Example 1 and Comparative Example A. For the deposition assay, each MDI tested, each MDI was actuated 45 times and then the canister with valve assembly was removed from the actuator housing. The canister was chilled by immersion in a liquid nitrogen bath for about one minute and the valve assembly was removed from the canister. The formulation was then poured from the opened canister into a separate receiving flask. The valve assembly was maintained at ambient conditions for 30 minutes to allow it to warm to ambient temperature (20-21° C. A glass boiling tube was filled with 50 mL of the collection solvent and a portion of the collection solvent was used to carefully wash (by pipet) the valve assembly so that the washings were captured in the boiling tube. The collection solvent was sodium dodecyl sulfate (SDS, 10 mM) in 60:40 (volume/volume) acetonitrile:50 mM ammonium acetate, pH5.5. The gasket was removed from the assembly and with a side-cut pliars the bottle emptier, metering chamber, and stem assembly were removed from the valve ferrule. All of the valve components (coated and uncoated) were added to the boiling tube. The tube was capped and then sonicated for two minutes followed by vigorous shaking by hand for one minute. An aliquot of the collection solvent was analyzed for sample content using an HPLC assay with references to known standards. An Agilent 1100 HPLC instrument (Agilent Technologies, Santa Clara, Calif.) with a UV detector (220 nm) and a symmetry shield RP-18, 4.6 mm-150 mm column (25° C. column temp) was used. Fresh collection solvent was used as the mobile phase. The flow rate was 1.0 mL/minute. The amounts of umeclidinium and vilanterol recovered from the valve components are reported in Table 1. Three individual MDIs (n=3) were tested and the results are presented as the mean values.

The results in Table 1 are reported based on the following equations:

$$umeclidinium recovered ( µg ) = [ umeclidinium bromide recovered ( µg ) ] \times [ MW umeclidinium \div MW umeclidinium bromide ] \\ vilanterol recovered ( µg ) = [ vilanterol trifenatate recovered ( µg ) ] \times [ MW vilanterol \div MW vilanterol trifenatate ]$$

TABLE 1
Valve Deposition Assay

| | Umeclidinium Recovered | Vilanterol Recovered |
|---|---|---|
| | from Valve Assembly | from Valve Assembly |
| | [micrograms with standard | [micrograms with standard |
| | deviation (SD)] | deviation (SD)] |
| Example 1 | 493.1 | 199.2 |
| | (SD = 49.2) | (SD = 19.2) |
| Comparative | 868.9 | 353.8 |
| Example A | (SD = 121.3) | (SD = 50.7) |

1. A pressurized canister comprising a formulation, the formulation comprising
one or more active pharmaceutical ingredients,
one or more propellants, and
PVP; wherein
the interior of the pressurized canister is coated with a coating selected from a poly(fluoroalkylene) polymer, a copolymer of poly(fluoralkylenes), and the condensation product of a first coating layer comprising a silane having one or more reactive silane groups and a second coating layer deposited on the first coating layer and comprising an at least partially fluorinated compound having at least one reactive silane group.

2. The pressurized canister of claim 1, wherein the one or more active pharmaceutical ingredients has at least one amine moiety and at least one alcohol moiety.

3. The pressurized canister of claim 1, wherein the one or more active pharmaceutical ingredients comprises umeclidinium or a pharmaceutically acceptable salt thereof, wherein the salt is optionally bromide, vilanterol or a pharmaceutically acceptable salt thereof, wherein the salt is optionally trifenatate, or a combination of the foregoing.

4. The pressurized canister of claim 1, wherein the PVP has a K-value of 10 and 95.

5. The pressurized canister of claim 1, wherein the PVP is about 0.005% to about 0.05% by weight of the formulation.

6. The pressurized canister of claim 1, wherein the coating comprises a copolymer of poly(fluoralkylenes).

7. The pressurized canister of claim 6, wherein the copolymer of poly(fluoroalkylenes) is a copolymer of a C2-C4 fluoroalkylene and a C3-C6 fluoroalkylene.

8. The pressurized canister of claim 6, wherein the copolymer of poly(fluoroalkylenes) is a copolymer of hexafluoropropylene and tetrafluoroethylene.

9. The pressurized canister of claim 1, wherein the coating comprises the condensation product of a second coating layer that is a perfluoropolyether silane according to Formula Ia in which R^{f} comprises from 20 to 40 linked repeating units
Formula Ia:
R^{f}[Q¹-[C(R)₂—Si(Y)₃₋ₓ(R^{1a})ₓ]_{y}]_{z}
wherein:
R^{f} is a monovalent or multivalent polyfluoropolyether moiety;
Q¹ is an organic divalent or trivalent linking group;
each R is independently hydrogen or a C1-4 alkyl group;
each Y is independently a hydrolysable group;
R^{1a} is a C1-8 alkyl or phenyl group;
x is 0 or 1 or 2;
y is 1 or 2; and
z is 1, 2, 3, or 4,
and a first coating layer comprising a silane having two or more reactive silane groups separated by an organic linker group.

10. The pressurized canister of claim 1, wherein the coating comprises the condensation product of a first coating layer comprising a silane polymer having one or more reactive silane groups and a second coating layer deposited on the first coating layer wherein the second coating layer comprises a polyfluoropolyether silane of the formula
R^{f}Q¹ᵥ[Q²_{w}-[C(R⁴)₂—Si(X)₃₋ₓ(R⁵)ₓ]_{y}]_{z}
wherein:
R^{f} is a polyfluoropolyether moiety;
Q¹ is a trivalent linking group;
each Q² is an independently selected organic divalent or trivalent linking group;
each R⁴ is independently hydrogen or a C₁₋₄ alkyl group;
each X is independently a hydrolysable or hydroxyl group;
R⁵ is a C₁₋₈ alkyl or phenyl group; and
v and w are independently 0 or 1, x is 0 or 1 or 2; y is 1 or 2; and z is 2, 3, or 4.
v and w are independently 0 or 1, x is 0 or 1 or 2; y is 1 or 2; and z is 2, 3, or 4.

11. The pressurized canister of claim 9, wherein Formula Ia is Formula Ib
Formula Ib
R^{f}[Q¹-[C(R)₂—Si(O—)₃₋ₓ(R^{1a})ₓ]_{y}]_{z}
wherein:
R^{f} is a monovalent or multivalent polyfluoropolyether segment;
Q¹ is an organic divalent or trivalent linking group;
each R is independently hydrogen or a C1-4 alkyl group; and
R^{1a} is a C1-8 alkyl or phenyl group.

12. The pressurized container of claim 1, wherein the formulation contains no more than a trace amount of any component other than the one or more active pharmaceutical ingredients, the one or more propellants, and the PVP.

13. The pressurized container of claim 12, further comprising a trace amount of water, alcohol, or water and alcohol.

14. An inhaler comprising the pressurized canister of claim 1.

15. The inhaler of claim 14, wherein the inhaler further comprises a valve and an actuator in communication with the valve.

16. The inhaler of claim 15, wherein the valve is a metering valve

17. The inhaler of claim 14, wherein at least a portion of the metering valve is coated with the poly(fluoroalkylene) polymer or a copolymer of poly(fluoralkylenes).

18. A method of actuating an inhaler comprising actuating the actuator of an inhaler of claim 15 for a sufficient time to release at least of a portion of the formulation from the pressurized canister.

19. A method of administering a formulation comprising actuating the actuator of an inhaler of claim 15 for a sufficient time to release at least a portion of the formulation from the pressurized container; and inhaling at least a portion of the formulation.

20. The method of claim 19, wherein the method comprises administering a pharmaceutically acceptable amount of umeclidinium, vilanterol, or a combination thereof.

21-33. (canceled)
